(19) United States
(12) Patent Application Publication
(10) Pub. No.: US 2026/0168943 A1
(43) Pub. Date: Jun. 18, 2026

(54) Artificial Intelligence-Based CT Metal Artifact Reduction Method for Multi-Physics Monitoring

(71) Applicant: Institute of Geology and Geophysics, Chinese Academy of Sciences, Beijing (CN)

(72) Inventors: Shaojiang WU, Beijing (CN); Yibo WANG, Beijing (CN)

(73) Assignee: Institute of Geology and Geophysics, Chinese Academy of Sciences, Beijing (CN)

(21) Appl. No.: 19/530,056

(22) Filed: Feb. 4, 2026

(30) Foreign Application Priority Data
Jul. 24, 2025 (CN) 202511023518.1

(51) Int. Cl. G01N 23/046; G01N 23/083; G01N 33/24

(52) U.S. Cl. CPC G01N 23/046; G01N 23/083; G01N 33/241; G01N 2223/401; G01N 2223/50

(57) ABSTRACT

The present invention provides an artificial intelligence-based method for reducing CT metal artifacts in multi-physics monitoring, and hydraulic fracturing operations. CT data with and without metal artifacts from piezoelectric ceramic sensors, and a location map of the piezoelectric ceramic sensors are used to construct an artifact reduction module based on a dual-domain three-channel approach. The artifact reduction module uses both image domain and chord diagram domain information to obtain CT data with the metal artifacts from piezoelectric ceramic sensors removed. The method can suppress CT metal artifacts and improve CT image quality, and thus obtain more accurate rock property information under specific pressure conditions. Reservoir mechanical characteristics can be inferred under specific construction conditions, such as wellhead injection pressure, providing guidance information for actual shale gas hydraulic fracturing reservoir field construction and management.

### TECHNICAL FIELD

This invention relates to the field of image data processing technology, specifically to “image enhancement or restoration,” and more particularly to an artificial intelligence-based multi-physics monitoring CT metal artifact reduction method.

### BACKGROUND

Hydraulic fracturing technology involves injecting high-pressure fluid into shale reservoirs to generate complex artificial fractures, which can increase reservoir connectivity and improve single-well production. Monitoring and evaluating the different stages of hydraulic fracturing reservoir stimulation is a prerequisite for achieving efficient development and safe production. However, in hydraulic fracturing field monitoring, the conditions of the field well site are complex, the arrangement range of the observation system is limited, and accurate background data of the actual working area (velocity structure, rock mechanics parameters, etc.) cannot be obtained. At the same time, there are many uncontrollable factors in actual operation. Therefore, it is difficult to verify the correctness of fracture inversion results during hydraulic fracturing through on-site monitoring.

Rock physics and hydraulic fracturing experiments can provide stable and controllable stress loading and signal measurement conditions. By preparing rock samples from actual shale gas reservoir outcrops or drill cores, accurate information on rock properties under specific pressure conditions can be obtained, which can then provide guidance for actual shale gas hydraulic fracturing reservoir operations.

Laboratory rock physics hydraulic fracturing experiments can perform multi-physics monitoring (ultrasonic, acoustic emission, CT). In laboratory rock physics experiments, multi-physics monitoring such as ultrasonic and acoustic emission can be performed simultaneously. Computed Tomography (CT) is an imaging technology that obtains information about the object under test in a non-destructive manner. CT imaging uses an excited CT X-ray beam to perform tomographic scanning of the rock sample, which can obtain medium structure information of the internal structure of the rock sample. Active source ultrasonic uses piezoelectric ceramic sensors (PZT) to excite seismic waves. The seismic waves propagate within the rock sample, and the medium information such as the velocity inside the rock sample can be obtained by inverting the received seismic waves. PZT passive source acoustic emission uses piezoelectric ceramic sensors to continuously and passively collect acoustic emission signals generated by internal rock damage. Source parameters related to internal rock fracture can be obtained through acoustic emission signal analysis. In these monitoring methods, piezoelectric ceramic sensors are installed on the rock surface and form a detector array in a certain manner. These sensors realize the switching of PZT receiving and transmitting functions through a fast automatic switching system, which can passively collect acoustic emission signals with PZT, and can also actively excite ultrasonic signals.

However, artifact problems exist during multi-physics monitoring with CT. The imaging process of the equipment is as follows: the CT X-ray tube makes a circular motion around the object X (the object to be measured), and exposure is performed when moving to different angles. The light tube emits X-rays, which are attenuated after being absorbed by the object, and the remaining X-rays reach the detector. The detector converts the received light intensity signal into an electrical signal. The signals received by all units on the detector form projection data X (projection image). The projection data generates the final image through an image reconstruction algorithm. The most commonly used reconstruction algorithm in the industry is the filtered back-projection algorithm. The characteristic of this CT algorithm is to convolve and filter the projection under each acquired projection angle before back-projection, compensating for high-frequency components, thereby improving the problem of blurred reconstructed images after direct back-projection, and the reconstructed image quality is better.

When there is a high-density object (such as metal) within the imaging field of view, striped and banded black and white artifacts will appear in the reconstructed image. The causes of metal artifacts are mainly: CT beam hardening, scattering effects, complete photon attenuation, and partial volume effects of metals. Among them, beam hardening is the main cause of metal artifacts. The rays generated by the X-ray tube have a certain spectral width, that is, they contain X-rays of different energy levels. When multi-energy X-rays pass through an object, low-energy rays are easily absorbed, and high-energy rays are more likely to pass through. The average energy of the received rays will become higher, and the rays will gradually become harder, which is called the beam hardening effect. When X-rays encounter a very high-density substance X (metal), the phenomenon of beam hardening will be aggravated, and the acquired projection data will change drastically in the metal area and the non-metal area, and metal artifacts will appear in the reconstructed image. CT

Compared to rock samples, PZT sensors have a higher density, similar to that of metals, which generates strong “metal artifacts” during CT scanning. These artifacts severely interfere with the interpretation of the internal images of the rock sample, preventing the acquisition of accurate information about the hydraulic fracturing rock properties, and thus hindering the provision of accurate guidance for on-site hydraulic fracturing reservoir modification operations.

At present, the related metal artifact reduction technologies mainly include the following methods: (1) Projection domain interpolation algorithm. The projection domain linear interpolation method is based on the similarity of linear attenuation coefficients of adjacent tissues (taking the human body as an example), that is, linear interpolation is used to fill the metal trajectory with the values near the metal trajectory. The projection domain interpolation method can effectively correct most metal artifacts by filling the metal projection trajectory. However, due to the instability of metal segmentation accuracy and the abrupt change of data at the edge of the metal implant trajectory, it is easy to cause secondary artifact problems, which also reduces the quality of the reconstructed image. (2) Iterative reconstruction algorithm. This method starts from an initial image and iteratively updates the reconstructed image by continuously reducing the error between the actual projection value and the theoretical projection value. The iterative reconstruction method mainly uses the non-missing projection data in the sinogram to reconstruct the image, and reconstructs the artifact-free image after a sufficient number of iterative updates. Due to its complicated algorithm steps and huge CT calculation volume, the iterative algorithm usually takes a long time to calculate. (3) Deep learning algorithm. Deep learning algorithms mainly include key steps such as feature learning, mapping relationship establishment, and model training. This method automatically learns potential feature patterns from the database, and establishes a mapping relationship from artifact-containing images to artifact-free images through training, thereby removing artifacts. It can be divided into supervised, unsupervised and semi-supervised methods. Deep learning-based MAR (Metal Artifact Reduction) methods are mainly divided into three categories: sinogram enhancement, image enhancement, CT, and joint dual enhancement of sinogram-CT images. Among them, joint dual enhancement methods based on sinograms and images, such as artifact removal methods based on the Unfolding CT idea, have achieved performance improvements, but the artifact removal effect is still not ideal due to the optimization algorithm and network structure design used.

### SUMMARY

The objective of this invention is to at least partially overcome the shortcomings of the prior art and provide an artificial intelligence-based multi-physics monitoring CT metal artifact reduction method, thereby providing guidance information for applied shale gas hydraulic fracturing reservoir transformation field construction.

Another objective of this invention is to provide an artificial intelligence-based multi-physics monitoring CT metal artifact reduction method that effectively solves the CT artifact problem existing in laboratory rock physics hydraulic fracturing experiments during multi-physics monitoring, thereby providing guidance information for actual shale gas hydraulic fracturing reservoir transformation field construction.

A further objective of this invention is to provide an artificial intelligence-based multi-physics monitoring CT metal artifact reduction method to improve image quality, thereby providing guidance information for shale gas hydraulic fracturing reservoir transformation field construction.

To achieve the above objectives or one of the above objectives, the technical solution of this invention is as follows:

An artificial intelligence-based multi-physics monitoring CT metal artifact reduction method, the method comprising:
Selecting rock samples prepared from actual shale gas reservoir outcrops or reservoir transformation drilling cores, conducting laboratory rock physics hydraulic fracturing experiments, and performing multi-physics monitoring;
Constructing a training dataset: the dataset includes CT data without piezoelectric ceramic sensor metal artifacts, CT data containing piezoelectric ceramic sensor metal artifacts, and piezoelectric ceramic sensor position maps, which are matched to form a data volume, with all data existing in the form of a data volume; the dataset consists of real rock sample experimental data and simulated synthetic data; data augmentation is performed to improve the generality of the dataset; all data are preprocessed using the same steps;
Constructing a dual-domain three-channel artifact reduction module, wherein the artifact reduction module uses two domain information: image domain and chord diagram domain; the first channel is constructed as an image domain-based UNet-type submodule, used to obtain the position information of the piezoelectric ceramic sensor and provide constraints for artifact reduction; the second channel is a UNet-type submodule with joint dual enhancement of image domain and chord diagram domain, used to suppress the metal artifact effects caused by the piezoelectric ceramic sensor; the third channel is a chord diagram domain channel; the UNet-type submodule of the first channel uses skip connections; the UNet-type submodule of the first channel and the UNet-type submodule of the second channel share piezoelectric ceramic sensor information through a cross-spatial attention mechanism to provide information constraints for the artifact reduction process;
Training the detection network using the training dataset;
Preprocessing all actually monitored data using the same steps, inputting the preprocessed CT data containing piezoelectric ceramic sensor metal artifacts into the artifact reduction module to obtain the corresponding CT data with piezoelectric ceramic sensor metal artifacts removed and the piezoelectric ceramic sensor position map;
Updating the dataset and the artifact reduction module. According to a preferred embodiment of the present invention, the step of “conducting laboratory rock physics hydraulic fracturing experiments and performing multi-physics monitoring” includes: Sampling rock samples of different specifications according to the research objectives, covering the rock samples with pre-fabricated rubber sleeves, which have multiple probe holes, and the holes are not yet fitted with piezoelectric ceramic sensors;
Using an laboratory hydraulic fracturing experimental device to acquire CT data of the rock samples without piezoelectric ceramic sensors. This CT data does not contain metal artifacts from the piezoelectric ceramic sensors and is used as the first stage CT data;
Placing the piezoelectric ceramic sensors into the probe holes and bonding the piezoelectric ceramic sensors to the surface of the rock samples;
Using the laboratory hydraulic fracturing experimental device to acquire CT data of the rock samples with the piezoelectric ceramic sensors attached, and using the CT data to calibrate the spatial position of the piezoelectric ceramic sensors. This CT data contains metal artifacts from the piezoelectric ceramic sensors and is used as the second stage CT data;
Applying different loading strategies to the rock samples. The loading stages include an isotropic loading stage, an increasing axial pressure stage, a water injection and pore pressure increase stage, and a pressure unloading stage; during the water injection and pore pressure increase stage, several sets of CT data are acquired at set time intervals. This CT data contains metal artifacts from the piezoelectric ceramic sensors and is used as the third stage CT data; during the pressure unloading stage, one set of CT data is acquired. This CT data contains metal artifacts from the piezoelectric ceramic sensors and is used as the fourth stage CT data; then, the piezoelectric ceramic sensors are removed from the rock samples, and another set of CT data is acquired. This CT data does not contain metal artifacts from the piezoelectric ceramic sensors and is used as the fifth stage CT data;
Processing all the acquired CT data. According to a preferred embodiment of the present invention, the specifications of the rock samples are: cylindrical shape with a diameter of 50 mm and a length of 125 mm;
The rock samples include two types: ordinary rock samples and interfering rock samples; The ordinary rock samples include two types of rocks: sandstone and shale; they are divided into 5 categories based on the presence and direction of bedding, namely bedding directions of 0±20°, 45±20°, 90±20°, 135±20°, and homogeneous medium without bedding; they are further divided into 2 categories based on axial compressive strength, namely 50±10 MPa and 90±10 MPa; 5 rock samples are selected for each category, totaling 100 rock samples;
Rock samples are prepared from actual shale gas reservoir outcrops or reservoir modification drilling cores. The interfering rock samples include two types of rocks: sandstone and shale, which are homogeneous media without bedding, and the rock samples contain sediments; 5 rock samples are selected for each type, totaling 10 rock samples.

According to a preferred embodiment of the present invention, the generation of real rock sample experimental data includes: Using ordinary rock samples and interfering rock samples, the first stage CT data, the second stage CT data, the fourth stage CT data, and the fifth stage CT data are collected to form the total dataset. According to a preferred embodiment of the present invention, in the “training the detection network using the training dataset” step, the dataset is divided into a training set and a test set in a ratio of 8:2; the artifact reduction module uses a stochastic gradient descent optimization method, with a dynamic learning rate, an initial value of 0.0001, reduced by half every 50 iterations, a batch size of 40, and 200 iterations; the training of the artifact reduction module is performed on a GPU image processing unit. According to a preferred embodiment of the present invention, the CT data containing metal artifacts from the piezoelectric ceramic sensor, the CT data without metal artifacts from the piezoelectric ceramic sensor, and the extracted piezoelectric ceramic sensor position map are updated to the training dataset when any of the following conditions are triggered:
The image similarity M_{I} between the current n-th image with artifacts Iₙ and the (n−1)-th image with artifacts Iₙ₋₁ is greater than 5%; or
The image similarity M_{I} between the current n-th artifact-removed image Iₙ and the (n−1)-th artifact-removed image Iₙ₋₁ is greater than 5%; or
The image similarity M_{Ide} between the current n-th piezoelectric ceramic sensor position map Iₙ and the (n−1)-th piezoelectric ceramic sensor position map Iₙ₋₁ is greater than 3%.

According to a preferred embodiment of the present invention, the update of the artifact reduction module is triggered when the following conditions are met:
The accumulated number of updated data volumes after the start of the experiment is greater than 5; or
The similarity difference between images Iₙ and Iₙ₋₁ is greater than 10%.

According to a preferred embodiment of the present invention, the positions of the probe holes on the rubber sleeve are: four columns are arranged along the circumferential direction of the rock sample at 0°, 90°, 180°, and 270°, with multiple probe holes evenly distributed in each column.

The height of the piezoelectric ceramic sensors in every two columns of the four columns of probe holes is different, so that the four columns of piezoelectric ceramic sensors do not appear at the same height simultaneously.

According to a preferred embodiment of the present invention, the laboratory hydraulic fracturing experimental device includes a pressure vessel, a loading system, an acoustic emission counting and waveform acquisition system, and a CT monitoring system.

According to a preferred embodiment of the present invention, during the process of pressurizing rock samples using different loading strategies, active source ultrasonic data is collected at set time intervals. During active source ultrasonic monitoring, some piezoelectric ceramic sensors act as transmitting probes to excite ultrasonic signals, while the remaining piezoelectric ceramic sensors act as receiving probes to receive the ultrasonic signals.

During the process of pressurizing rock samples using different loading strategies, the piezoelectric ceramic sensors, in addition to being used as transmitting and receiving probes during active source ultrasonic data acquisition, also act as receiving probes to receive acoustic emission signals generated by changes in the rock sample at other times.

According to a preferred embodiment of the present invention, the data processing of CT data includes: CT imaging, reduction of piezoelectric ceramic sensor metal artifacts, and joint analysis with acoustic emission results.

According to a preferred embodiment of the present invention, the data processing of acoustic emission data includes: effective event picking, first arrival picking, source localization, source mechanism analysis, magnitude calculation, and stress analysis.

According to a preferred embodiment of the present invention, the data processing of ultrasonic data includes: identification of ultrasonic events, ultrasonic first arrival picking, and velocity inversion analysis.

The artificial intelligence-based multi-physics monitoring CT metal artifact reduction method of this invention utilizes an adaptive AI network update mechanism to dynamically suppress PZT metal artifacts in CT images, improving the accuracy of CT image analysis and obtaining more accurate rock property information under specific pressure conditions. This, in turn, allows for a more accurate inference of reservoir mechanical characteristics under specific construction conditions, such as wellhead injection pressure, providing guidance for applied shale gas hydraulic fracturing reservoir field operations.

### BRIEF DESCRIPTION OF THE DRAWINGS

FIG. 1 shows the arrangement of the rock sample and piezoelectric ceramic sensors according to an embodiment of the present invention, with the rock sample on the left and the arrangement of PZT on the surface of the rock sample on the right;

FIG. 2 shows the modular architecture of the multi-physics monitoring CT metal artifact reduction method according to an embodiment of the present invention, mainly including two parts: the CT image PZT artifact reduction part and the module update part; and

FIG. 3 is a schematic diagram of the structure of the artifact reduction module according to an embodiment of the present invention.

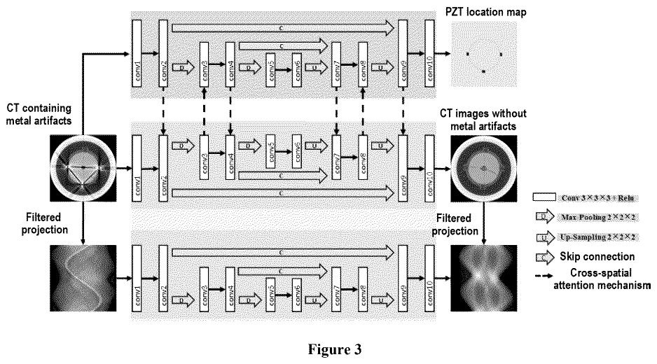

### DETAILED DESCRIPTION OF EMBODIMENTS

The exemplary embodiments of the present invention are described in detail below with reference to the accompanying drawings, where the same or similar reference numerals represent the same or similar elements. Furthermore, in the following detailed description, for ease of explanation, numerous specific details are set forth to provide a thorough understanding of the disclosed embodiments. However, it will be apparent that one or more embodiments may be practiced without these specific details. In other instances, well-known structures and devices are shown in diagrammatic form to simplify the drawings.

As mentioned earlier, rock samples were prepared from field samples of shale gas reservoir outcrops or reservoir stimulation drilling cores. During laboratory rock physics hydraulic fracturing experiments, data from CT, ultrasound, and acoustic emission were simultaneously collected, enabling multi-physics monitoring of the laboratory rock physics hydraulic fracturing experiment. However, during the CT data acquisition process, the PZT sensors used for ultrasound and acoustic emission signal acquisition, due to their high density, produced strong “metal artifacts.” These artifacts severely interfered with the interpretation and analysis of the internal images of the rock samples, reducing the research value of the multi-physics monitoring.

This application proposes an artificial intelligence network that simultaneously utilizes image domain and chord diagram domain constraints to suppress “metal artifacts” caused by PZT probes in CT images during rock physics experiments. Specifically, a one-channel image-domain-based UNet-type submodule is constructed to obtain the position information of the PZT sensors and provide constraints for artifact reduction. A two-channel image-domain and chord-diagram-domain joint dual-enhanced UNet-type submodule is constructed to suppress the metal artifact effects caused by the PZT probes. This invention uses different types of rock samples (rock types, different directional layering, different axial compressive strengths), and attaches PZT probes to the surface of the rock samples for CT scanning to construct a training set. During the testing process, an adaptive data update mechanism and module update mechanism based on the difference analysis between two adjacent images are designed. The method proposed in this invention can dynamically suppress PZT metal artifacts, improve the accuracy of CT image analysis, and obtain more accurate rock property information under specific pressure conditions. This allows for a more accurate inference of reservoir mechanical characteristics under specific construction conditions, such as wellhead injection pressure, providing guidance for actual shale gas hydraulic fracturing reservoir stimulation field operations.

Artificial Intelligence (AI) is the theory, methods, techniques, and application systems that use digital computers or machines controlled by digital computers to simulate, extend, and expand human intelligence, perceive the environment, acquire knowledge, and use knowledge to obtain optimal results. Machine Learning (ML) is a multidisciplinary field involving probability theory, statistics, approximation theory, convex analysis, algorithm complexity theory, and many other disciplines. This field of study focuses on how computers can simulate or implement human learning behavior to acquire new knowledge or skills, and reorganize existing knowledge structures to continuously improve their performance. Machine learning is the core of artificial intelligence and the fundamental way to enable computers to possess intelligence. Its applications span various fields of artificial intelligence. Machine learning and deep learning typically include techniques such as artificial neural networks, belief networks, reinforcement learning, transfer learning, inductive learning, and learning by demonstration.

The following describes the specific process of the multi-physics monitoring CT metal artifact reduction method based on artificial intelligence, which is an embodiment of the present invention.

1. Basic Information on the Rock Physics Experiments Involved in this Invention

1.1 Experimental Sample Preparation Experimental samples are typically obtained from actual shale gas reservoir outcrops or core samples from reservoir stimulation wells. The size of the rock samples can be varied according to the actual analytical objectives. The specifications used in conventional laboratory hydraulic fracturing experiments are: cylindrical samples with a diameter of 50 mm and a length of 125 mm.

Two types of rock samples are used:

Normal rock samples and interfering rock samples: Normal rock sample types include: rock type (2 types, such as sandstone and shale), with different bedding orientations (bedding directions approximately 0±20°, 45±20°, 90±20°, 135±20°, and homogeneous media without obvious bedding, 5 types), and different axial compressive strengths (50±10 MPa, 90±10 MPa, 2 types);

5 samples are selected for each type, totaling 100 rock samples.

Interfering rock sample types include: rock type (2 types, such as sandstone and shale), homogeneous media without obvious bedding, and the rock samples contain obvious sediments, such as heavy mineral impurities and detrital particles. These sediments are internal characteristics of the rock sample and will be visible in CT images regardless of whether PZT probes are attached to the surface, increasing the universality of the rock samples. 5 samples are selected for each type, totaling 10 rock samples.

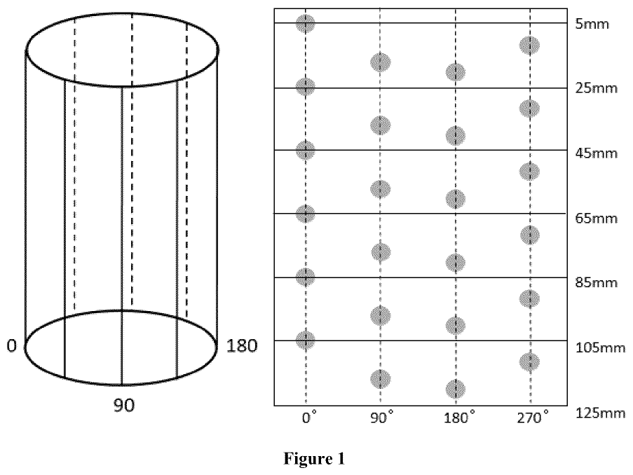

A pre-fabricated rubber sleeve is used to cover the rock sample. The rubber sleeve has 24 PZT probe holes (not limited to 24, this number depends on the design of the rubber sleeve). The PZT probes are placed in the holes, and the bottom of the PZT probes are glued to the surface of the rock sample. The positions of the PZT probe holes on the rubber sleeve are shown in FIG. 1, arranged in four columns along 0°, 90°, 180°, and 270° of the rock sample. The height of the probes in each column is different, ensuring that no two probes appear at the same height simultaneously. Since the PZT probes are made of metal, they will produce serious metal artifacts on the rock sample surface, affecting the CT imaging quality of the rock sample; this, in turn, hinders the provision of guidance information for shale gas hydraulic fracturing reservoir field operations. The inventive scheme will reduce the metal interference caused by multiple PZT probes at any given height (as shown in FIG. 1).

### 1.2 Experimental System Configuration:

The experimental system is a laboratory hydraulic fracturing experimental device, mainly composed of a pressure vessel, a loading system, and an acoustic emission counting and waveform acquisition system.
(1) The pressure vessel is a high-pressure resistant metal container, in which the prepared rock sample is placed.
(2) The loading system generally uses a triaxial compression stress loading system. This system can provide axial pressure, injection pressure, and confining pressure to the inside of the pressure vessel, used to simulate the in-situ formation conditions of the rock sample.
(3) The acoustic emission counting and waveform acquisition system consists of PZT sensors, preamplifiers, and high-speed acquisition equipment.
(4) The CT monitoring system consists of a CT scanner, meeting a field of view greater than 125 mm, with a scanning mode of planar scanning, usually a 256-slice CT scanner.

### 1.3 Experimental Procedure:

The experimental system can adopt different loading strategies according to the actual research objectives. The experimental procedure of this patent is as follows:
(1) Place the rock sample in a rubber sleeve and load it into the pressure vessel; place it on the CT scanner and acquire CT data once. The CT data collected in this process does not contain interference from PZT metal artifacts.
(2) Remove the rock sample from the container, and attach PZT sensors (or PZT probes) to the PZT sensor holes in the rubber sleeve outside the rock sample.
(3) Place the rock sample with attached PZT sensors (or PZT probes) into the pressure vessel; place it on the CT scanner and acquire CT data once. The CT data collected in this process contains interference from PZT metal artifacts. Calibrate the spatial position Sta(x,y,z) of all sensors. (Because the PZT sensor hole diameter is larger than the sensor diameter, there will be spatial deviations when installing the PZT sensors in each experiment, so it is necessary to accurately extract the position through CT images after installing the PZT sensors).
(4) Isotropic loading stage (confining pressure).
(5) Increasing axial pressure stage (axial pressure).
(6) Water injection to increase pore pressure stage (injection pressure), until the rock fracture stage and unloading stage. During this process, the CT scanner is turned on, and CT data is collected according to the set time. The CT data collected in this process contains interference from PZT metal artifacts.
(7) Pressure unloading stage (injection pressure, confining pressure, and axial pressure are unloaded to 0): CT data is acquired once. The CT data acquired during this process includes interference from PZT metal artifacts (after the rock sample fractures, it contains internal cracks). The rock sample is removed from the container, and the PZT probes attached to the PZT probe holes in the rubber sleeve outside the rock sample are removed. The rock sample is then placed back into the pressure vessel and placed on the CT scanner, where CT data is acquired again. The CT data acquired during this process does not include interference from PZT metal artifacts.

During processes (4)-(6) above, active source ultrasonic data is collected according to the set time. During active source ultrasonic monitoring, some PZT probes act as transmitting probes (excitation probes) to excite ultrasonic signals, while the remaining probes act as receiving probes to receive ultrasonic signals.

During processes (4)-(6) above, due to the application of external force, acoustic emission events will occur. Except when acting as transmitting probes during active source ultrasonic acquisition, the PZT probes act as receiving probes for the rest of the time, receiving acoustic emission signals generated by changes in the rock sample.

### 1.4, Main Data Analysis Content:

The CT data processing flow includes: data acquisition, CT imaging, PZT metal artifact reduction, and joint analysis with acoustic emission results.

The acoustic emission data processing flow includes: effective event picking (picking data segments with clear phases), first arrival picking (extracting the first arrival time of the waveform in the effective event data), source location (obtaining the source location of the event based on the picked first arrival time and the spatial position of the probes), source mechanism analysis (inverting the source mechanism of the event based on the waveform of the event to obtain the fracture characteristics of the event), magnitude calculation (inverting the magnitude of the event based on the waveform of the event), stress analysis (based on the waveform of the event or the source mechanism of the event), etc.

The ultrasonic data processing flow includes: identification of ultrasonic events (identifying the signals received by other channels based on the ultrasonic excitation time), ultrasonic first arrival picking (obtaining the first arrival time of the received signal), and velocity inversion analysis (inverting the velocity model of the rock sample based on the first arrival time, waveform, and other information).

### 2. Constructing the Training Dataset:

### 2.1 Dataset Overview.

The dataset includes: CT data without PZT metal artifacts, CT data with PZT metal artifacts, and PZT position maps (binary images where the PZT probe position is set to 1 and all other positions are set to 0). These three components are matched and correspond to each other, forming a data volume. All data exists in the form of data volumes. The dimensions of all the above data are consistent; the data space acquired in a single CT scan is (N₁, N₂, N₃); in this experiment, there are a total of Nₘ pairs, and the total data volume is Nₘ(N₁, N₂, N₃, 3).

### 2.2 Dataset Generation.

The dataset consists of real rock sample experimental data and simulated synthetic data.

### (1) Real Rock Sample Data Generation:

Using the ordinary rock samples prepared in 1.1, complete the experimental process in 1.3.

Repeat this experiment Nₖ times using different ordinary rock samples from 1.1 to form the total dataset. In the k-th experiment, select one set of CT data from processes 1.3(1) and 1.3(3) respectively. Extract the PZT position from the CT data containing PZT metal interference to obtain the PZT position map. Calibrate the obtained CT images to form a data volume; this data volume is acquired before fracturing and does not contain fracturing cracks.

In the k-th experiment, select two sets of CT data from process 1.3(7). Extract the PZT position from the CT data containing PZT metal interference to obtain the PZT position map. Calibrate the obtained CT images to form a data volume; this data volume is acquired after fracturing, and the rock damage contains fracturing cracks.

Using the interference rock samples prepared in 1.1, complete the experimental process in 1.3. Repeat this experiment N_k times using different interference rock samples from 1.1 to form the total dataset.

In the k-th experiment, CT data was acquired once each using processes 1.3(1) and 1.3(3). The PZT positions were extracted from the CT data containing PZT metal interference to obtain a PZT position map. The acquired CT images were then calibrated to form a data volume; this data volume was acquired before fracturing and does not contain fracturing cracks.

In the k-th experiment, two sets of CT data from process 1.3(7) were selected. The PZT positions were extracted from the CT data containing PZT metal interference to obtain a PZT position map. The acquired CT images were then calibrated to form a data volume; this data volume was acquired after fracturing, and its rock damage contains fracturing cracks.

### (2) Synthetic Rock Sample Data Generation:

According to the same dimensions as the real rock sample, and referencing the image characteristics of the real rock samples (shale, sandstone) in (1), CT images (CT data) of synthetic rock samples were generated; referencing the real rock sample experiments in (1), the fracturing characteristics (crack width, extension morphology, etc.) generated in fracturing process 1.3(6) were added to the above synthetic CT images (CT data) to form a synthetic rock sample containing cracks.

The CT image characteristics of the rubber sleeve were added to the synthetic rock sample, forming a synthetic rock sample without PZT probes; the CT image characteristics of the PZT probes were then added to form a synthetic rock sample containing PZT probes.

Based on the above synthetic rock samples, CT images without PZT metal artifacts and CT images with PZT metal artifacts were obtained. The PZT positions were extracted from the CT data containing PZT metal interference to obtain a PZT position map, and the above images were combined to form a data volume.

This process was repeated to obtain multiple data volumes.

### 2.3, Data Augmentation.

During the data generation process, data augmentation was performed to improve the generality of the dataset. The data augmentation method is as follows:

The CT data obtained in step 1.3(1) of the interference rock sample experiment described in section 2.2, which does not contain PZT metal artifacts, is used. The parts of this data containing significant interference, such as heavy mineral impurities and detrital particles, are extracted to form noise images. These extracted noise images are then randomly added to the data volume (not added to the PZT location map), maintaining consistent addition locations (including relative positions to the PZT probe), quantity, and amplitude within the data volume. The number of added interfering elements (0 to 5, randomly distributed) and their amplitude (−2 to 2, randomly distributed) vary in each individual data volume.

### 2.4, Data Preprocessing.

All data undergoes the same preprocessing steps.

### 3. Constructing the PZT Artifact Reduction Method:

### 3.1. Module Architecture.

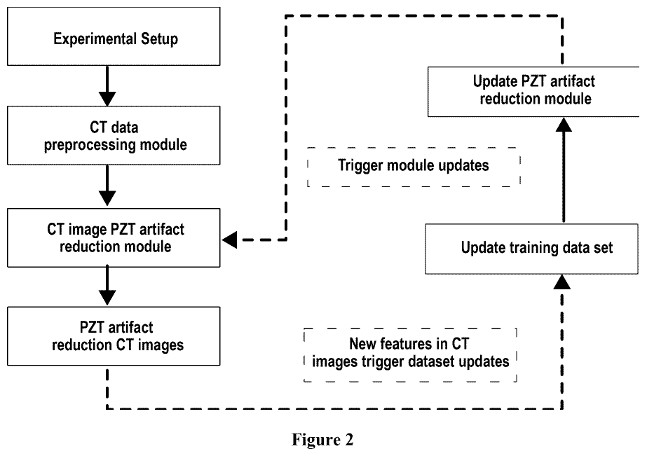

The multi-physics monitoring CT metal artifact reduction method is shown in FIG. 2, and includes the experimental setup, multi-physics data acquisition module (CT data preprocessing module), CT image PZT artifact reduction module, parameter update module, and PZT artifact-suppressed CT image.

The experimental setup and multi-physics data acquisition module mainly include the experimental part in 1.3, corresponding to that section.

The CT image PZT artifact reduction module contains the main structure of the PZT artifact reduction method, mainly described in section 3.2.

The parameter update module mainly includes fine-tuning and updating the CT image PZT artifact reduction module based on the updated training dataset.

### 3.2. CT Image PZT Artifact Reduction Module

A dual-domain three-channel artifact reduction module is constructed, which uses two types of domain information: the image domain and the sinogram domain. A single-channel UNet-type submodule based on the image domain is constructed to obtain the PZT position information and provide constraints for artifact reduction. A two-channel image domain and sinogram domain joint dual-enhanced UNet-type submodule is constructed to suppress the metal artifacts caused by the PZT probe.

In the single-channel PZT position information extraction UNet-type submodule:

The image domain channel uses 10 convolutional layers (Conv units). The parameters of the 1-10 convolutional layers are: (256×3×3×3), (256×3×3×3), (128×3×3×3), (128×3×3×3), (64×3×3×3), (64×3×3×3), (128×3×3×3), (128×3×3×3), (256×3×3×3), (256×3×3×3). The first dimension is the number of convolutions, and the last three dimensions are the convolution size.

In the two-channel PZT artifact reduction UNet-type submodule:

The image domain channel uses 10 convolutional layers. The parameters for convolutional layers 1-10 are: (256×3×3×3), (256×3×3×3), (128×3×3×3), (128×3×3×3), (64×3×3×3), (64×3×3×3), (128×3×3×3), (128×3×3×3), (256×3×3×3), (256×3×3×3). The first dimension represents the number of convolutions, and the last three dimensions represent the convolution size.

The chord diagram domain channel uses 10 convolutional layers. The parameters for convolutional layers 1-10 are: (256×3×3×3), (256×3×3×3), (128×3×3×3), (128×3×3×3), (64×3×3×3), (64×3×3×3), (128×3×3×3), (128×3×3×3), (256×3×3×3), (256×3×3×3). The first dimension represents the number of convolutions, and the last three dimensions represent the convolution size. In the PZT position information extraction UNet-type submodule, skip connections are used to make PZT position information extraction more accurate.

The PZT position information extraction UNet-type submodule and the PZT artifact reduction UNet-type submodule share PZT information through a cross-spatial attention mechanism to provide information constraints for the artifact reduction process.

During training, the left input terminal receives CT images without PZT artifact interference, and the right output terminal receives CT images containing PZT artifact interference and PZT position maps; the loss function is MSE (mean squared error). The total loss function is MSE=MSE1+MSE2; where MSE1 is the error between the artifact-suppressed CT image calculated using the CT image containing PZT metal interference and the CT image without PZT artifact interference; MSE2 is the error between the PZT position image (piezoelectric ceramic sensor position map) calculated using the CT image containing PZT metal interference and the true PZT position image.

### 4. Training the Detection Network Using the Training Dataset

4.1, The dataset is divided into a training set and a test set, with a ratio of 8:2.

4.2, This PZT artifact reduction module uses the stochastic gradient descent optimization method; a dynamic learning rate is set, with an initial value of 0.0001, decreasing by half every 50 iterations. The batch size is set to 40; the number of iterations is 200.

4.3, The training of the PZT artifact reduction module is performed on a GPU image processing unit.

### 5. Analysis and Processing of Actual Monitoring Data:

### 5.1 Data Preprocessing.

Rock samples were prepared from shale gas reservoir outcrops or drill core samples. All monitoring data underwent the same preprocessing steps.

### 5.2 Data Analysis.

The preprocessed CT images containing PZT metal artifacts are input into the PZT artifact reduction module to obtain the corresponding CT images with PZT metal artifacts removed and the PZT location map.

### 5.3 Dataset Update Mechanism

When any of the following conditions are met, the input image containing PZT metal artifacts, the image after artifact removal, and the extracted PZT location are updated to the training dataset:
(1) Image similarity analysis is performed between the current n-th image with artifacts Iₙ and the (n−1)-th image with artifacts Iₙ₋₁, obtaining the image similarity M_{I} between two consecutive images (n-th and (n−1)-th) with artifacts when this value is greater than 5%.
(2) Image similarity analysis is performed between the current n-th artifact-removed image Iₙ and the (n−1)-th artifact-removed image Iₙ₋₁, obtaining the image similarity M_{Ide} between two consecutive artifact-removed images (n-th and (n−1)-th) when this value is greater than 5%.
(3) Image similarity analysis is performed between the current n-th PZT location map Iₙ and the (n−1)-th PZT location map Iₙ₋₁, obtaining the image similarity M_{Ide} between two consecutive PZT location maps (n-th and (n−1)-th) when this value is greater than 3%.

The image similarity calculation formula is, where N1, N2, and N3 are the dimensions of the three-dimensional image, and i, j, and k are the indices for traversing the image:

$$M = \frac{1}{{N}_{1} {N}_{2} {N}_{3}} {\sum }_{i = 1}^{N 1} {\sum }_{j = 1}^{N 2} {\sum }_{k = 1}^{N 3} {[ {I}_{n} ( i , j , k ) - {I}_{n - 1} ( i , j , k ) ]}^{2}$$

### 6. Parameter Update Module and Parameter Update:

6.1 Module Update Mechanism. The module triggers an update when the following conditions are met.
(1) After the experiment begins, the cumulative number of updated data samples is greater than 5.
(2) In any trigger condition of 5.3, the similarity difference between two adjacent images (i.e., images Iₙ and Iₙ₋₁) is greater than 10%.

### 6.2, Parameter Update Method.

Based on the parameters of the aforementioned PZT artifact reduction module, the newly added data after the start of this experiment, i.e., the updated data in 5.3 (both data sets are still Nₖ, maintaining the same quantity), are used to fine-tune the PZT artifact reduction module, improving training speed and maintaining update efficiency.

### 6.3, Updating PZT Artifact Reduction Module Parameters.

The artificial intelligence-based multi-physics monitoring CT metal artifact reduction method of this invention utilizes an adaptive AI network update mechanism to dynamically suppress PZT metal artifacts in CT images, improving the accuracy of CT image analysis and obtaining more accurate rock property information under specific pressure conditions. This, in turn, allows for a more accurate inference of reservoir mechanical characteristics under specific construction conditions, such as wellhead injection pressure, providing guidance for actual shale gas hydraulic fracturing reservoir stimulation field operations.

Although embodiments of the present invention have been shown and described, it will be understood by those skilled in the art that various changes may be made to these embodiments without departing from the principles and spirit of the invention. The scope of the invention is defined by the appended claims and their equivalents.

1. An artificial intelligence-based multi-physics monitoring CT metal artifact reduction method, comprising conducting laboratory rock physics hydraulic fracturing experiments and performing multi-physics monitoring as follows:
constructing a training dataset comprising real rock sample experimental data and simulated synthetic data, wherein the dataset includes CT data without metal artifacts from piezoelectric ceramic sensors, CT data containing metal artifacts from piezoelectric ceramic sensors, and a position map of the piezoelectric ceramic sensors, wherein the three datasets are matched and corresponding to form a data volume, with all data existing in the form of a data volume; augmenting the data to improve the generality of the dataset; and performing the same preprocessing steps on all data;
constructing a dual-domain three-channel artifact reduction module using information from two domains: an image domain and a chord diagram domain; wherein a first channel is constructed as an image-domain-based UNet-type submodule, and is used to obtain the position information of the piezoelectric ceramic sensors and provide constraints for artifact reduction; wherein a second channel is a jointly dual-enhanced UNet-type submodule in the image domain and chord diagram domain, used to suppress the metal artifact effects caused by the piezoelectric ceramic sensors; and wherein a third channel is a chord diagram domain channel; and wherein the UNet-type submodule of the first channel uses skip connections; and the UNet-type submodule of the first channel and the UNet-type submodule of the second channel share piezoelectric ceramic sensor information through a cross-spatial attention mechanism to provide information constraints for the artifact reduction process;
training the detection network using the training dataset by performing the same preprocessing steps on all actual monitoring data, inputting the preprocessed CT data containing metal artifacts from piezoelectric ceramic sensors into the artifact reduction module to obtain the corresponding CT data with the metal artifacts from piezoelectric ceramic sensors removed and the piezoelectric ceramic sensor position map; and updating the dataset and the artifact reduction module.

2. The multi-physics monitoring CT metal artifact reduction method based on artificial intelligence according to claim 1, characterized in that the step of “conducting laboratory rock physics hydraulic fracturing experiments and performing multi-physics monitoring” includes:
sampling rock samples of different specifications according to the research objectives, and covering the rock samples with pre-fabricated rubber sleeves having multiple probe holes, wherein the holes are configured to be fitted with piezoelectric ceramic sensors;
collecting CT data of the rock samples without piezoelectric ceramic sensors using an laboratory hydraulic fracturing experimental device, wherein this CT data does not contain metal artifacts from piezoelectric ceramic sensors and is used as the first stage CT data;
placing piezoelectric ceramic sensors into the probe holes and bonding them to the surface of the rock samples;
collecting CT data of the rock samples with the attached piezoelectric ceramic sensors using the laboratory hydraulic fracturing experimental device, and using the CT data to calibrate the spatial position of the piezoelectric ceramic sensors, wherein the CT data of the rock samples with the attached piezoelectric ceramic sensors contains metal artifacts from the piezoelectric ceramic sensors and is used as the second stage CT data;
applying different loading strategies to the rock samples, wherein pressurization stages include an isotropic loading stage, an increasing axial pressure stage, a water injection increasing pore pressure stage, and a pressure unloading stage; wherein the water injection stage comprises an increasing pore pressure stage; and collecting several sets of CT data at set time intervals, wherein the CT data contains metal artifacts from the piezoelectric ceramic sensors and is used as the third stage CT data; wherein during the pressure unloading stage, CT data is collected once, and wherein this CT data contains metal artifacts from the piezoelectric ceramic sensors and is used as the fourth stage CT data; then, removing the piezoelectric ceramic sensors from the rock samples and collecting another set of CT data, wherein this CT data does not contain metal artifacts from the piezoelectric ceramic sensors and is used as the fifth stage CT data; and,
processing all the collected CT data.

3. The artificial intelligence-based multi-physics monitoring CT metal artifact reduction method according to claim 2, characterized in that:
the rock samples have the following specifications: diameter of 50 mm, length of 125 mm, cylindrical shape;
the rock samples include two types: ordinary rock samples and interfering rock samples;
wherein the ordinary rock samples include two types of rocks: sandstone and shale; they are divided into 5 categories based on the presence and direction of bedding, namely bedding directions of 0±20°, 45±20°, 90±20°, 135±20°, and homogeneous medium without bedding; they are further divided into 2 categories based on axial compressive strength, namely 50±10 MPa and 90 10 MPa; and five rock samples are selected from each category, totaling 100 rock samples;
wherein the interfering rock samples include two types of rocks: sandstone and shale, which are homogeneous media without bedding, and the rock samples contain sediments; and five rock samples are selected from each category, totaling 10 rock samples.

4. The artificial intelligence-based multi-physics monitoring CT metal artifact reduction method according to claim 3, characterized in that the generation of real rock sample experimental data includes:
collecting rock samples from shale gas reservoir outcrops or reservoir drilling holes, comprising ordinary rock samples and interfering rock samples, and collecting the first-stage CT data, second-stage CT data, fourth-stage CT data, and fifth-stage CT data to form the total dataset.

5. The artificial intelligence-based multi-physics monitoring CT metal artifact reduction method according to claim 1, characterized in that:
In the “training the detection network using the training dataset” step, the dataset is divided into a training set and a test set with a ratio of 8:2; the artifact reduction module uses a stochastic gradient descent optimization method, with a dynamic learning rate, an initial value of 0.0001, reduced by half every 50 iterations, with a batch size of 40, and 200 iterations; wherein the training of the artifact reduction module is performed on a GPU image processing unit.

6. The artificial intelligence-based multi-physics monitoring CT metal artifact reduction method according to claim 1, characterized in that, when any of the following conditions are triggered, the CT data containing metal artifacts from the piezoelectric ceramic sensor, the CT data without metal artifacts from the piezoelectric ceramic sensor, and the extracted piezoelectric ceramic sensor position map are updated to the training dataset as a data volume:
when the image similarity M_{I} between the current n-th artifact-containing image Iₙ and the (n−1)-th artifact-containing image Iₙ₋₁ is greater than 5%; or
when the image similarity M_{Ide} between the current n-th artifact-removed image Iₙ and the (n−1)-th artifact-removed image Iₙ₋₁ is greater than 5%; or
when the image similarity M_{Ide} between the current n-th piezoelectric ceramic sensor position map Iₙ and the (n−1)-th piezoelectric ceramic sensor position map Iₙ₋₁ is greater than 3%.

7. The artificial intelligence-based multi-physics monitoring CT metal artifact reduction method according to claim 6, characterized in that, the update of the artifact reduction module is triggered when the following conditions are met:
the accumulated number of updated data volumes after the start of the experiment is greater than 5; or the similarity difference between images Iₙ and Iₙ₋₁ is greater than 10%.

8. The artificial intelligence-based multi-physics monitoring CT metal artifact reduction method according to claim 2, characterized in that:
the positions of the probe holes on the rubber sleeve are: four columns are arranged along the circumferential direction of the rock sample at 0°, 90°, 180°, and 270°, with multiple probe holes evenly distributed in each column, and the height of the piezoelectric ceramic sensors in every two columns of the four columns of probe holes is different, so that the four columns of piezoelectric ceramic sensors do not appear at the same height simultaneously.

9. The artificial intelligence-based multi-physics monitoring CT metal artifact reduction method according to claim 2, characterized in that:
the laboratory hydraulic fracturing experimental device includes a pressure vessel, a loading system, an acoustic emission counting and waveform acquisition system, and a CT monitoring system.

10. The artificial intelligence-based multi-physics monitoring CT metal artifact reduction method according to claim 2, characterized in that:
during the process of pressurizing the rock sample using different loading strategies, active source ultrasonic data is collected at set times; during active source ultrasonic monitoring, some piezoelectric ceramic sensors act as transmitting probes to excite ultrasonic signals, while the remaining piezoelectric ceramic sensors act as receiving probes to receive the ultrasonic signals;
during the process of pressurizing the rock sample using different loading strategies, the piezoelectric ceramic sensors, in addition to being used as transmitting and receiving probes during active source ultrasonic data acquisition, also act as receiving probes to receive acoustic emission signals generated by changes in the rock sample at other times.
